(19) United States
(12) Patent Application Publication
(10) Pub. No.: US 2014/0329332 A1
(43) Pub. Date: Nov. 6, 2014

(54) DEVICE AND METHOD FOR RAPID ASSAY OF MULTIPLE BIOLOGICAL SAMPLES FOR OXYGEN CONSUMPTION

(71) Applicants: Dmitri Boris Papkovsky, Blarney County Cork (IE); James Nial Hynes, Ovens (IE)

(72) Inventors: Dmitri Boris Papkovsky, Blarney County Cork (IE); James Nial Hynes, Ovens (IE)

(73) Assignee: LUXCEL BIOSCIENCES LTD., Cork (IE)

(21) Appl. No.: 14/360,153

(22) PCT Filed: Nov. 22, 2011
(86) PCT No.: PCT/EP2011/070648

§ 371 Date: Jun. 25, 2014

(51) Int. Cl. G01N 33/00

(52) U.S. Cl. CPC G01N 33/0004
USPC 436/136; 422/83

(57) ABSTRACT

Device (10) for rapid detection of biological oxygen consumption and method of classifying biological samples (e.g., positive and negative samples) (5) by detecting biological oxygen consumption. Each well (29) on a multi-well plate (20, 30) is hermetically sealed from the surrounding environment and other wells after deposit of a biological sample within the well by a cover plate (30) and an adherent gasket (40) sandwiched between the plates.

### BACKGROUND

A wide range of biological samples, ranging from foods susceptible to carrying foodborne pathogens to agricultural seeds, are evaluated by a variety of different techniques to determine whether the sample is positive (i.e., biologically active) or negative (i.e., biologically inactive).

Current techniques, while generally effective for establishing whether a biological sample is positive or negative, are time-consuming and cumbersome.

Hence, a substantial need exists for a technique and a device capable of elegant, reliable and early classification of a biological sample as a positive or negative sample.

### SUMMARY OF THE INVENTION

A first aspect of the invention is a sensitive device for rapid detection of

biological oxygen consumption. The device includes an oxygen impermeable base plate, an oxygen impermeable cover plate, a gasket and a plurality of oxygen sensitive photoluminescent probes. The base plate has an array of open-top wells. The cover plate is configured and arranged to overlay the base plate so as to cover the open top of the wells in the base plate. The gasket is configured and arranged for placement between the base plate and the cover plate, and operable for hermetically separating each individual well when the cover plate overlays the base plate. The plurality of oxygen sensitive photoluminescent probes are configured and arranged on the device so that a probe will be in fluid communication with each individual hermetically sealed well.

A second aspect of the invention is a method of monitoring consumption of

oxygen by biological samples employing the device of the first aspect of the invention.

A first embodiment of the second aspect of the invention includes the steps of (i) obtaining a device in accordance with the first aspect of the invention, (ii) placing a biological sample into each of a plurality of the wells in the device, (iii) compressing the gasket between the cover plate and the base plate so as to hermetically seal each individual well, (iv) incubating the biological samples hermetically sealed within the wells, and (v) interrogating each probe in communication with a hermetically sealed well containing a biological sample with an interrogation device wherein interrogations measure changes in the probe reflective of changes in oxygen concentration within the well.

A second embodiment of the second aspect of the invention includes the steps of (i) obtaining a device in accordance with the first aspect of the invention, (ii) placing a biological sample into each of a plurality of the wells in the device, (iii) heat sealing the gasket to the cover plate and the base plate so as to hermetically seal each individual well, (iv) incubating the biological samples hermetically sealed within the wells, and (v) interrogating each probe in communication with a hermetically sealed well containing a biological sample with an interrogation device wherein interrogations measure changes in the probe reflective of changes in oxygen concentration within the well.

### BRIEF DESCRIPTION OF THE DRAWINGS

FIG. 1 is top view of one embodiment of a device in accordance with this invention with the cover plate in the open position and the release liner partially removed to facilitate viewing of components otherwise obscured by the release liner.

FIG. 2 is an enlarged cross-sectional side view of a portion of the invention depicted in FIG. 1 just prior to positioning of the cover plate into the closed position overlaying the base plate.

### DETAILED DESCRIPTION OF A PREFERRED EMBODIMENT

### Definitions

As used herein, including the claims, the term “fluid” includes any substance that has no fixed shape, yields easily to external pressure and is capable of flowing, including gases and liquids.

As used herein, including the claims, the phrase “oxygen permeable” means a material that when formed into a 1 mil film has an oxygen transmission rate of greater than 1,000 c³/m² day when measured in accordance with ASTM D 3985.

As used herein, including the claims, the phrase “oxygen impermeable” means a layer of material or laminated layers of materials that has an oxygen transmission rate of less than 100 cm³/m² day when measured in accordance with ASTM F 1927.

As used herein, including the claims, the phrase “oxygen barrier” means a layer of material or laminated layers of materials that has an oxygen transmission rate of less than 200 cm³/m² day when measured in accordance with ASTM F 1927.

As used herein, including the claims, the phrase “oxygen impervious”, when used to describe a hermetically sealed well, means limiting passage of oxygen into or out from the hermetically sealed well, at a T of 25° C., a ΔP of 0 and a ΔO₂ concentration of 20%, such that the O₂ concentration within the hermetically sealed well changes less than 1% per day (e.g., a change from 2% O2 to 2.5% O₂ within a hermetically sealed well over a 28 hour period is oxygen impervious (a 0.5% change over more than a 24 hour period) while a change from 100%) to 96% O₂ within a hermetically sealed well over a 24 hour period is not oxygen impervious (a 4% change)).

As used herein, including the claims, the phrase “manually puncturable with a blunt tipped object”, means having a level 0 puncture resistance performance rating when measured in accordance with Standard EN388.

As used herein, including the claims, the phrase “classified sample”, means a sample sorted according to a sample characteristic as between two mutually exclusive classes (e.g., positive and negative samples).

As used herein, including the claims, the phrase “graded sample”, means a sample sorted according to a sample characteristic as between three or more classes (e.g., grade A, grade B, grade C and grade D samples).

As used herein, including the claims, the phrase “positive sample” means a sample that, when in operable communication with a probe sensitive to a given variable and within any appropriate test period during which pertinent changes in the given variable

As used herein, including the claims, the phrase “negative sample” means a sample that, when in operable communication with a probe sensitive to a given variable and after any appropriate test period during which pertinent changes in the given variable should occur in the sample, does not cause the probe to generate a perceptible signal indicating that the value of the given variable possessed or exhibited by the sample is significant, as evidenced by a failure to reach a detectable threshold value (e.g., an absolute value, a rate of change value, etc.). For example, (i) a sample in communication with an oxygen sensitive photoluminescent probe in which viable aerobic bacteria or a germinating seed have not consumed sufficient oxygen to reduce the oxygen concentration in the sample below 10% O₂ from the initial concentration within an appropriate test period of twelve hours—detected by measuring changes in the probe's optical signal—when the threshold value is at 10% O₂ from the initial concentration, is a negative sample, and (ii) a sample in communication with an oxygen sensitive photoluminescent probe in which viable aerobic bacteria or a germinating seed required sixteen hours to consume sufficient oxygen to reduce the oxygen concentration in the sample to a threshold value of a 10% reduction from the initial concentration—detected by measuring changes in the probe's optical signal—when the appropriate test period is twelve hours, is a negative sample.

### Nomenclature

10 Device
20 Base Plate
29 Wells in Base Plate
30 Cover Plate
40 Gasket
50 Release Liner
60 Probes
70 Live Hinge Connecting Cover Plate to Base Plate
80 Latch
S Biological Sample (e.g., a quiescent seed)
W Water

### Description

Construction

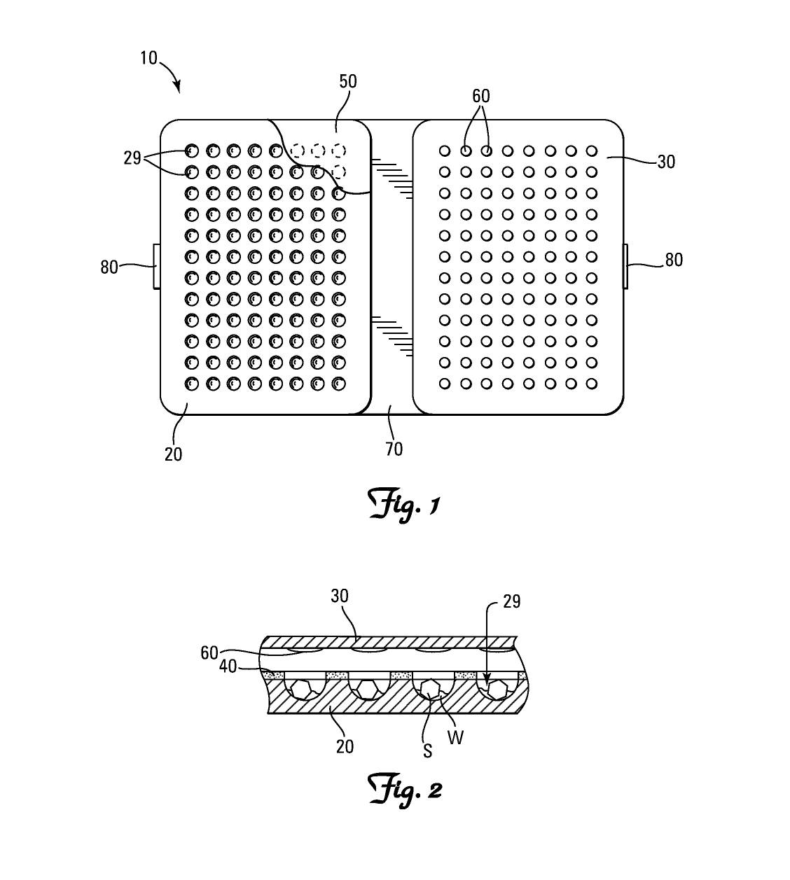

Referring to FIGS. 1 and 2, a first aspect of the invention is a device 10 effective for rapidly, accurately and reliably classifying or grading a biological sample S (e.g., a positive or negative sample S, or a class A, B, C or D sample S) by detecting biological oxygen consumption. The device 10 includes an oxygen impermeable base plate 20, an oxygen impermeable cover plate 30, a gasket 40 and a plurality of oxygen sensitive photoluminescent probes 60.

The base plate 20 has an array of open-top wells 29, such as a standard 6, 12, 24, 48, 96 or 384 well plate. The base plate 20 may be constructed from any oxygen impervious material, such as polyethylene terephthalate (PET), glycol-modified polyethylene terephthalate (PETG), polyvinyl chloride (PVC), polymethyl methacrylate (PMMA), glass, etc. The base plate 20 is preferably an oxygen barrier and most preferably oxygen impermeable. One option for construction of the base plate 20 is lamination of a first relatively thick perforated layer of material (not shown) onto a second relatively thin layer of material (not shown) wherein the perforations in the first layer form the wells.

The wells 29 are preferably sized to accommodate the desired boilogical samples S (e.g., food sample or seed) with minimal remaining headspace, in order to limit the total amount of O₂ in the hermetically sealed well 29 upon commencement of testing and thereby allow consumption of even a relatively small quantities of O₂ by a biological sample S produces a detectable change in O₂ concentration within the well 29.

The base plate 20 can be transparent, translucent or opaque. The base plate 20 is preferably transparent to allow visual confirmation of a positive or negative classification.

The cover plate 30 is configured and arranged to overlay the base plate 20 so as to cover the open top of the wells 29 in the base plate 20. As with the base plate 20, the cover plate 30 may be constructed from any oxygen impervious material, such as polyethylene terephthalate (PET), glycol-modified polyethylene terephthalate (PETG), polyvinyl chloride (PVC), polymethyl methacrylate (PMMA), glass, aluminum, etc. The cover plate 30 is preferably an oxygen barrier and most preferably oxygen impermeable.

The base plate 20 and cover plate 30 can be provided as separate alignable components, or as depicted in FIG. 1 they can be conveniently attached to one another by a live hinge 70 or other suitable pivotable connecting mechanism. A latch 80 is also preferably provided for ensuring a firm and secure attachment of the plates 20 and 30. When provided as separate alignable components, the plates 20 and 30 are preferably provided with alignment couplings (e.g., pin and pin hole, tab and channel, peripheral shoulders, etc.) to ensure proper alignment of the plates 20 and 30 when they are overlaid and attached to one another.

The gasket 40 is configured and arranged for placement between the base plate 20 and the cover plate 30, and operable for both (i) securing the cover plate 30 to the base plate 20, and (ii) hermetically separating each individual well 29 in the base plate 20 once the cover plate 30 is secured to the base plate 20.

So long as the thickness of the gasket 40 is less than about 5 mil, the gasket 40 can but need not be constructed from an oxygen impervious material. This is because the surface area of the gasket 40 exposed to each well 29 (i.e., circumference of the well 29 times the thickness of the gasket 40) is small, the distance from the edge of one well 29 to the edge of an adjacent well 29 (i.e., the “thickness” of the gasket 40 across which permeating oxygen must traverse) is fairly large, and the ΔP_{O2} between wells 29 is likely to be small.

The gasket 40 can be an adhesive, such as a pressure sensitive adhesive, a contact adhesive, a heat-activated adhesive, a radiation-cured adhesive, a moisture-cured adhesive, etc. Generally, acrylic and silicone pressure sensitive adhesives are preferred. Alternatively, the gasket 40 can be a heat-sealable thermoplastic or coated foil.

When the gasket 40 is a pressure sensitive adhesive, the exposed surface(s) of the gasket 40 is preferably covered with a release liner 50.

The gasket 40 can be provided as a separate and independent layer, or it can be laminated onto the inner major surface of either the base plate 20 or the cover plate 30. When provided as a separate and independent layer or laminated to the cover plate 30, the gasket 40 can be provided as a continuous sheet or film that covers the open top of the wells 29, or a perforated sheet or firm with openings that align with the wells 29 so as to prevent the gasket 40 from covering the wells 29. When laminated to the base plate 20, as shown in FIG. 2, the gasket 40 is perforated with openings that align with the wells 29 in order to allow biological samples S to be deposited into the wells 29.

The cover plate 30 and if necessary the gasket 40 can be configured and arranged for detachment from the base plate 20 at any time after the wells 29 are hermetically sealed to permit removal of samples S from the wells 29.

The oxygen sensitive photoluminescent probes 60 are configured and arranged on the device 10 so that a probe 60 will be in fluid communication with each individual hermetically sealed well 29.

The probes 60 can be any device capable of sensing and reporting changes in oxygen concentration within an enclosed volume. In a preferred embodiment, the probes 60 are optically-active, oxygen sensitive materials configured and arranged to experience changes in oxygen concentration or partial pressure of oxygen P_{O2} in each well 29. The oxygen-sensitive material is preferably a photoluminescent dye embedded within an analyte permeable polymer matrix.

Solid-state polymeric materials based on oxygen-sensitive photoluminescent indicator dyes, are widely used as optical sensors and probes. See, for example U.S. Published Patent Applications 2009/0029402, 2008/8242870, 2008/215254, 2008/199360, 2008/190172, 2008/148817, 2008/146460, 2008/117418, 2008/0051646, 2006/0002822 and 2004/0033575, U.S. Pat. Nos. 7,569,395, 7,534,615, 7,368,153, 7,138,270, 6,689,438, 5,718,842, 4,810,655, and 4,476,870, and International Application 2009/128998. Such optical sensors are available from a number of suppliers, including Presens Precision Sensing, GmbH of Regensburg, Germany, Oxy sense of Dallas, Texas, United States, and Luxcel Biosciences, Ltd of Cork, Ireland.

The oxygen-sensitive photoluminescent dye may be selected from any of the well-known oxygen sensitive photoluminescent dyes. One of routine skill in the art is capable of selecting a suitable dye based upon the nature of the intended biological sample S to be tested. A nonexhaustive list of suitable oxygen sensitive photoluminescent dyes includes specifically, but not exclusively, ruthenium(II)-bipyridyl and ruthenium(II)-diphenylphenanothroline complexes, porphyrin-ketones such as platinum(II)-octaethylporphine-ketone, platinum(II)-porphyrin such as platinum(II)-tetrakis(pentafluorophenyl)porphine, palladium(II)-porphyrin such as palladium(II)-tetrakis(pentafluorophenyl)porphine, phosphorescent metallocomplexes of tetrabenzoporphyrins, chlorins, azaporphyrins, and long-decay luminescent complexes of iridium(III) or osmium(II).

Typically, the hydrophobic oxygen-sensitive photoluminescent dye is compounded with a suitable oxygen-permeable and hydrophobic carrier matrix. Again, one of routine skill in the art is capable of selecting a suitable oxygen-permeable hydrophobic carrier matrix based upon the nature of the intended biological sample S to be tested and the selected dye. A nonexhaustive list of suitable polymers for use as the oxygen-permeable hydrophobic carrier matrix includes specifically, but not exclusively, polystryrene, polycarbonate, polysulfone, polyvinyl chloride and some co-polymers. An alternative is to stain oxygen-permeable micro-beads with an oxygen-sensitive photoluminescent dye, mix the stained beads with silicone or polyurethane, and applying the mixture as a polymeric coating.

When the probes 60 are based on the quenching of photoluminescence by oxygen, an excitation and emission window (not shown) must be provided through the base plate 20, the cover plate 30 and/or the gasket 40 so that radiation at the excitation and emission wavelengths to be transmitted to and received from the probes 60 may pass with minimal interference. It is generally preferred to construct the entire plate 20 and/or 30 carrying the probes 60 from a material that allows radiation at the excitation and emission wavelengths to be transmitted to and received from the probes 60 with minimal interference. The probes 60 may be coated onto the base plate 20 within each well 29, as an array on the cover plate 30 so that a probe 60 rests atop and in fluid communication with each well 29 when the cover plate 30 is secured atop the base plate 20, or as an array on the gasket 60 so that a probe 60 rests atop and in fluid communication with each well 29 when the gasket 60 is laminated to the base plate 20.

The radiation emitted by an excited probe 60 can be measured in terms of intensity and/or lifetime (rate of decay, phase shift or anisotropy), with measurement of lifetime generally preferred as a more accurate and reliable measurement technique.

Instruments (not shown) for interrogating probes 60 based on the quenching of photoluminescence by oxygen are well known and commercially available from various sources, including bioMérieux SA of France and Mocon, Inc. of Minneapolis, Minn.

Use

The device 10 can be used to quickly, easily, accurately and reliably measure the partial pressure of oxygen within a hermetically sealed well 29 by (A) obtaining a device 10, (B) placing biological samples S into the wells 29 on the device 10 along with any culture medium (e.g., water W when the biological sample S is a seed), (C) hermetically sealing the wells 29 by (i) compressing or pressure sealing the gasket 60 between the cover plate 30 and the base plate 20 when the gasket 60 is a pressure sensitive or contact adhesive, (ii) compressing or pressure sealing the gasket 60 between the cover plate 30 and the base plate 20 and curing or activating the gasket 60 when the gasket 60 is a curable or activatable adhesive (e.g., heat-activated, radiation cured, moisture-cured, etc.), or (iii) heat sealing the gasket 60 when the gasket 60 is a heat sealable thermoplastic, (D) incubating the biological samples S retained within the hermetically sealed wells 29, and (E) interrogating each probe 60 in communication with a hermetically sealed well 29 containing a biological sample S with an interrogation device (not shown) wherein interrogations measure a state of the probe 60 reflective of oxygen concentration within the well 29.

Emissions read from each probe 60 can be converted to an oxygen concentration within the associate well 29 based upon a known conversion algorithm or look-up table. The probes 60 are preferably periodically interrogated throughout a testing period.

The device 10 can be used to quickly, easily, accurately and reliably measure changes in the partial pressure of oxygen within each hermetically sealed well 29 over time and thereby determine whether a biological sample S within the well 29 is positive or negative. Briefly, consumption of oxygen by a biological sample S employing the device 10 includes the steps of (A) obtaining a device 10, (B) placing biological samples S into the wells 29 on the device 10 along with any culture medium (e.g., water W when the biological sample S is a seed), (C) hermetically sealing the wells 29 by (i) compressing or pressure sealing the gasket 60 between the cover plate 30 and the base plate 20 when the gasket 60 is a pressure sensitive or contact adhesive, (ii) compressing or pressure sealing the gasket 60 between the cover plate 30 and the base plate 20 and curing or activating the gasket 60 when the gasket 60 is a curable or activatable adhesive (e.g., heat-activated, radiation cured, moisture-cured, etc.), or (iii) heat sealing the gasket 60 when the gasket 60 is a heat sealable thermoplastic, (D) incubating the biological samples S retained within the hermetically sealed wells 29, (E) ascertaining oxygen concentration within each hermetically sealed well 29 over time by (i) exposing the probe 60, at least once and preferably repeatedly, to excitation radiation over time, (ii) measuring radiation emitted by the excited probe 60 after at least one of the exposures, (iii) measuring passage of time during the excitation exposures and emission measurements, and (iv) converting at least one of the measured emissions to an oxygen concentration based upon a known conversion algorithm, and (F) reporting at least one of (i) at least two oxygen concentrations (one of which can be a known initial concentration and at least one of which is an ascertained concentration) and the time interval between those reported concentrations, and (ii) a rate of change in oxygen concentration within the hermetically sealed well 29 calculated from data obtained in step (E). Conversion algorithms used to convert the measured emissions to an oxygen concentration are well know to and readily developable by those with routine skill in the art.

Conversion of the data to an oxygen concentration can be skipped and the raw data used in many instances when the oxygen concentration need not be reported, such as when the report simply indicates whether the sample is a positive sample or a negative sample.

The radiation emitted by the excited probe 60 can be measured in terms of intensity and/or lifetime (rate of decay, phase shift or anisotropy), with measurement of lifetime generally preferred as a more accurate and reliable measurement technique when seeking to establish the extent to which the indicator dye has been quenched by oxygen.

1. A sensitive device for rapid detection of biological oxygen consumption comprising:
(a) an oxygen impermeable base plate having an array of open-top wells,
(b) an oxygen impermeable cover plate configured and arranged to overlay the base plate so as to cover the open top of the wells in fee base plate,
(c) a gasket configured and arranged for placement between the base plate and the cover plate and effective for hermetically separating each individual well when the cover plate overlays the base plate, and
(d) a plurality of oxygen sensitive photoluminescent probes configured and arranged on the device whereby a probe will be in fluid communication with each individual hermetically sealed well.

2. The device of claim 1 further comprising a means for aligning the cover plate onto the base plate.

3. The device of claim 1 wherein the cover plate is detachable from the base plate subsequent to hermetic sealing of the wells whereby samples may be removed from the wells.

4. The device of claim 1 wherein the cover plate is manually puncturable with a blunt-tipped object.

5. The device of claim 1 wherein at least the base plate is transparent.

6. The device of claim 1 wherein the base plate and cover plate are transparent.

7. The device of claim 2 wherein the device is a clamshell container whereby the cover plate is pivotable about a live hinge relative to the base plate as between an open position with the covet plate pivoted away from the base plate and a closed position with the cover plate overlaying the base plate.

8. The device of claim 7 further comprising a latch for securing the cover plate onto the base plate and compressing the gasket between the cover plate and the base plate when the cover plate is pivoted into the closed position.

9. The device of claim 1 wherein each hermetically sealed well is oxygen impervious.

10. (canceled)

11. (canceled)

12. The device of claim 1 wherein the gasket is a layer of pressure sensitive adhesive.

13. The device of claim 12 further comprising a release liner covering the layer of pressure sensitive adhesive.

14. The device of claim 1 wherein the gasket is a layer of heat sealable thermoplastic.

15. The device of claim 1 wherein file gasket is attached to the base plate.

16. The device of claim 2 wherein the gasket is attached to the cove plate.

17. The device of claim 15 wherein the gasket surrounds bat does not cover the open top of each individual well in the base plate.

18. The probe of claim 16 wherein the gasket forms a continuous layer over the surface of the cover plate.

19. (canceled)

20. (canceled)

21. The device of claim 2 wherein the probes are attached to the cover plate or to the base plate.

22. (canceled)

23. (canceled)

24. (canceled)

25. A method of monitoring consumption of oxygen by biological samples, comprising the steps of:
(a) obtaining a device in accordance with claim 1,
(b) placing a biological sample into each of a plurality of the wells in the device,
(c) compressing the gasket between the cover plate and the base plate so as to hermetically seal each individual well,
(d) incubating the biological samples hermetically sealed within the wells, and
(e) interrogating each probe in communication with a hermetically sealed well containing a biological sample with an interrogation device wherein interrogations measure changes in the probe reflective of chances in oxygen concentration within the well.

26. (canceled)

27. (canceled)

28. (canceled)

29. The method of claim 25, wherein the method further includes the step of puncturing the cover plate subsequent to interrogation so as to provide access to a well containing an incubated sample, and removing the incubated sample from the accessible well.

30. A method of monitoring consumption of oxygen by biological samples, comprising the steps of:
(a) obtaining a device in accordance with claim 14,
(b) placing a biological sample into each of a plurality of the wells in the device,
(c) heat sealing the gasket to the cover plate and the base plate so as to hermetically seal each individual well,
(d) incubating the biological samples hermetically sealed within the wells, and
(e) interrogating each probe in communication with a hermetically sealed well containing a biological sample with an interrogation device wherein interrogation measure changes in the probe reflective of changes in oxygen concentration within the well.

31. (canceled)

32. (canceled)

33. (canceled)

34. (canceled)
